(19) United States
(12) Patent Application Publication
(10) Pub. No.: US 2009/0018356 A1
(43) Pub. Date: Jan. 15, 2009

(54) Crystalline polymorph of exemestane

(75) Inventors: WeiYu Chen, Yongkang City (TW); SinChi Wang, Zhushan Town (TW); Shu-Ping Chen, Kaohsiung City 807 (TW)

Correspondence Address: COHEN, PONTANI, LIEBERMAN & PAVANE LLP, 551 FIFTH AVENUE, SUITE 1210, NEW YORK, NY, 10176, US

(73) Assignee: ScinoPharm Taiwan Ltd., Tainan County 741 (TW)

(21) Appl. No.: 12/214,957

(22) Filed: Jun. 24, 2008

Related U.S. Application Data
(60) Provisional application No. 60/937,099, filed on Jun. 25, 2007.

(51) Int. Cl. C07J 1/00

(52) U.S. Cl. 552/623

(57) ABSTRACT

New crystalline polymorph of exemestane characterized by a powder X-ray diffraction pattern having peaks at 10.7±0.1, 15.9±0.1, and 18.1±0.1 2-theta degree.

### RELATED APPLICATIONS

This application claims priority from U.S. Provisional Patent Application Ser. No. 60/937,099 which was filed on Jun. 25, 2007. The entire content of Provisional Patent Application Ser. No. 60/937,099 is incorporated herein as reference.

### BACKGROUND OF THE INVENTION

1. Field of the Invention

The invention relates to a novel crystalline polymorph of exemestane.

2. Description of the Related Art

Exemestane (brand name Aromasin®) is reported to be endowed with an aromatase-inhibiting action. Exemestane is chemically described as 6-methylenandrosta-1,4-diene-3,17-dione. Its molecular formula is C₂₀H₂₄O₂ and its structural formula is as follows

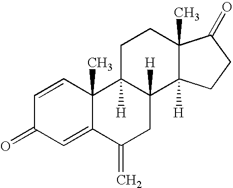

Various synthetic routes for making exemestane is known in the art.

There is still a need for developing a new form of exemestane, which is more suitable for pharmaceutical use.

### SUMMARY OF THE INVENTION

The present application invention provides a novel crystalline polymorph of exemestane and process of making the same.

In accordance with one embodiment of the present invention, the novel crystalline exemestane is characterized by a powder X-ray diffraction pattern having peaks at 10.7±0.1, 15.9±0.1, and 18.1±0.1 2-theta degree. Preferably, the powder X-ray diffraction pattern further has peaks at 17.5±0.1, 20.9±0.1, and 23.4±0.1 2-theta degree. More preferably, the powder X-ray diffraction pattern further has peaks at 16.4±0.1, 14.0±0.1, 14.4±0.1, 21.4±0.1, 22.9±0.1, 23.1±0.1, 26.1±0.1, and 29.3±0.1 2-theta degree.

In accordance with another embodiment of the present invention, the crystalline solid exemestane has a powder X-ray diffraction pattern as depicted in FIG. 1.

In accordance with yet another embodiment of the present invention, the crystalline solid exemestane has an infrared spectrum with bands at 2944±2 cm⁻¹, 1732±2 cm⁻¹, and 1659±2 cm⁻¹. Preferably, the infrared spectrum additionally has bands at 3078±2 cm⁻¹, 1623±2 cm⁻¹, 1406±2 cm⁻¹, 1298±2 cm⁻¹, 1003±2 cm⁻¹, 902±2 cm⁻¹, and 818±2 cm⁻¹.

As a preferred embodiment of the present invention, the crystalline solid exemestane has an infrared spectrum as depicted in FIG. 2.

The present application also provides a process of making crystalline solid exemstane comprising:
(1) dissolving crude exemestane with a solvent selected from the group consisting of acetone, ethanol, and mixture thereof to form a solution;
(2) forming crystals of exemestane by adding isopropyl ether to the solution of step 1) to obtain a slurry;
(3) filtering the slurry of step (2) to obtain the crystalline solid exemstane.

Preferably, the dissolving is carried out at a temperature of 70-80 Celsius degree. The step 2) is preferably conducted at a temperature of 0-10 Celsius degree.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIG. 1 shows an X-ray powder diffraction pattern of the solid crystalline exemestane in accordance with one embodiment of the present invention.

FIG. 2 shows an infrared spectrum of the solid crystalline exemestane in accordance with one embodiment of the present invention.

### DETAILED DESCRIPTION OF THE PRESENTLY PREFERRED EMBODIMENTS

The following examples are provided for illustrating, but not for limiting, of the present invention.

### Example 1

To a suitable reactor is charged Exemestane (about 3 g) and acetone (about 15 mL). The mixture is stirred and warmed up to 45-55° C. until the solid is dissolved. Isopropyl Ether (about 50 mL) is charged at 45-70° C. The solution is cooled to 0-10° C. and kept at 0-10° C. for NLT 1 hour. The slurry is filtered and dried at 70° C. to obtain about 0.4 g of Exemestane.

### Example 2

To a suitable reactor is charged Exemestane (about 3 g), EtOH (about 12 mL). The resulting mixture is stirred and warmed up to 70-80° C. until dissolved. Isopropyl Ether (about 72 mL) is charged at 60-80° C. the solution is cooled to 0-10° C. and kept at 0-10° C. for NLT 1 hour. The slurry is filtered and dried to obtain about 2.01 g of Exemestane.

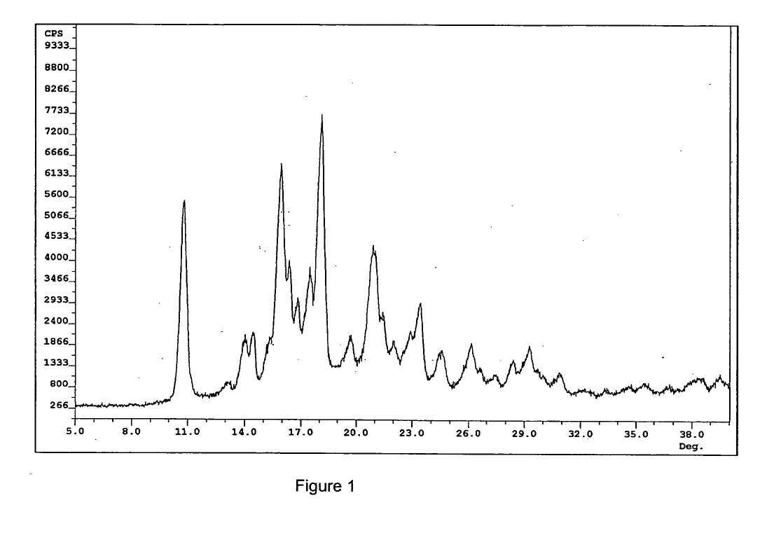

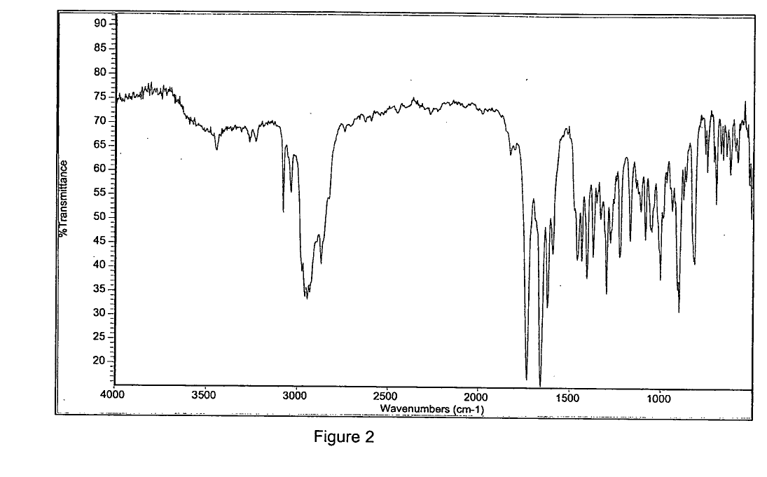

The above two processes (A) and (B) produce the same polymorph, which exhibits a X-ray powder diffraction pattern as shown in FIG. 1 and infrared spectrum as shown in FIG. 2.

The procedure of XRD test used for obtaining Figure is as follows. The test sample was milled and homogenously put on the tray of the X-ray machine, Scintag X2 Advance Diffraction, tested at continuous scan rate of 2.00 Deg/min, with range 5.00-40.00 (Deg.) and at a wavelength of 1.540562.

The procedure of IR test used for obtaining FIG. 2 is as follows. We weighed about 3 mg of sample and disperse the sample homogenously in 300 mg dry KBr, and then, immediately recorded the spectrum between 400 to 4000 cm⁻¹ by diffuse reflectance. We performed a single test on the sample. The IR machine was Nicolet, Magna-IR 560 Spectrometer. The number of sample scans was 32. The number of background scans was 32. The resolution was 4. The sample gain was 8. The mirror velocity was 0.6329. The aperture was 100.

The invention is not limited by the embodiments described above which are presented as examples only but can be modified in various ways within the scope of protection defined by the appended patent claims.

We claim:

1. A crystalline exemestane characterized by a powder X-ray diffraction pattern having peaks at 10.7±0.1, 15.9±0.1, and 18.1±0.1 2-theta degree.

2. The crystalline solid exemestane of claim 1 further having peaks in the powder X-ray diffraction pattern at 17.5±0.1, 20.9±0.1, and 23.4±0.1 2-theta degree.

3. The crystalline solid exemestane of claim 1 further having peaks in the powder X-ray diffraction pattern at 16.4±0.1, 14.0±0.1, 14.4±0.1, 21.4±0.1, 22.9±0.1, 23.1±0.1, 26.1±0.1, and 29.3+0.1 2-theta degree.

4. The crystalline solid exemestane of claim 1 further having a powder X-ray diffraction pattern as depicted in FIG. 1.

5. The crystalline solid exemestane of claim 1 having an infrared spectrum with bands at 2944±2 cm⁻¹, 1732±2 cm⁻¹, and 1659±2 cm⁻¹.

6. The crystalline solid exemestane of claim 5 further having an infrared spectrum with bands at 3078±2 cm⁻¹, 1623±2 cm⁻¹, 1406±2 cm⁻¹, 1298±2 cm⁻¹, 1003±2 cm⁻¹, 902±2 cm⁻¹, and 818±2 cm⁻¹.

7. The crystalline solid exemestane of claim 5 having an infrared spectrum as depicted in FIG. 2.

8. A process of making crystalline solid exemstane comprising:
(1) dissolving crude exemestane with a solvent selected from the group consisting of acetone, ethanol, and mixture thereof to form a solution;
(2) forming crystals of exemestane by adding isopropyl ether to the solution of step 1) to obtain a slurry;
(3) filtering the slurry of step (2) to obtain the crystalline solid exemstane.

9. The process of claim 8 wherein the solvent is acetone.

10. The process of claim 8 wherein the solvent is ethanol.

11. The process of claim 8 wherein the dissolving is carried out at a temperature of 70-80 Celsius degree.

12. The process of claim 8 wherein the step 2) is conducted at a temperature of 0-10 Celsius degree.
